(19) United States
(12) Patent Application Publication
(10) Pub. No.: US 2020/0251204 A1
(43) Pub. Date: Aug. 6, 2020

(54) INTEGRATED HEALTHCARE PERFORMANCE ASSESSMENT TOOL FOCUSED ON AN EPISODE OF CARE

(71) Applicant: KONINKLIJKE PHILIPS N.V., EINDHOVEN (NL)

(72) Inventors: Douglas Henrique Teodoro, São Paulo (BR); Niels Roman Rotgans, Eindhoven (NL); Lucas de Melo Oliveira, Melrose, MA (US); Daniel Robert Elgort, New York, NY (US)

(21) Appl. No.: 15/769,756

(22) PCT Filed: Oct. 25, 2016
(86) PCT No.: PCT/IB2016/056393

§ 371 Date: Apr. 20, 2018

Related U.S. Application Data
(60) Provisional application No. 62/248,479, filed on Oct. 30, 2015.

(51) Int. Cl. G16H 40/20; G06Q 10/06; G16H 50/70; G16H 10/60; G06Q 30/02; G06Q 10/10; G16H 50/20

(52) U.S. Cl. CPC G16H 40/20; G06Q 10/06393; G16H 50/70; G16H 50/20; G06Q 30/0205; G06Q 10/10; G06Q 10/067; G16H 10/60

(57) ABSTRACT

A health care performance assessment apparatus (10) includes at least one processor (16, 20, 22, 24) programmed to: collect information associated with medically-related events from a plurality of databases; group the collected information associated with medically-related events into episode of care (EOC) data structures wherein each EOC data structure contains the collected information for a single EOC treating a medical condition or providing a medical procedure to a single patient; store the EOC data structures in an EOC repository (18); group EOC data structures having a chosen commonality into at least one cohort; and calculate at least one key performance indicators (KPIs) for the cohorts. A display (2) is configured to display the KPIs for at least one selected cohort.

### FIELD

The following relates generally to the healthcare performance assessment arts and related arts.

### BACKGROUND

In the past decade, Electronic Medical Records (EMRs) have evolved from patient health information management systems, where demographics, clinical and administrative information were stored and retrieved for patients, to become complex enterprise resource planning systems that operate over the entire healthcare system. They include related services, such as administration, finance, supplier management, human resources, and decision support. They also generate subsidies for billing and reimbursement of service providers, and serve as a base for organizational support and cost management of healthcare facilities. Some EMRs provide academic functionality to support clinical research, epidemiological studies and information sharing between multi-professional teams. Thus, there are several examples of non-medical functionality that have been integrated into the EMR and are considered essential by many healthcare enterprises.

The availability of a large amount of patient records in the EMR, together with administrative, operational, and financial information, enables integrated key performance indicator (KPI) analytics across the hospital's patient population. KPI analytics provide tools for monitoring and assessing clinical effectiveness, patient safety, efficiency, staff orientation, and governance for quality improvement in the healthcare settings. Using metrics provided by KPIs, decisions can be made in the enterprise to improve clinical, operational and financial management. For example, when occupation rate data is available to decision makers in a timely manner, actions can be taken to increase or decrease capacity and staffing levels. Similarly, if the time between sepsis identification and antibiotic administration is known, actions can be taken to reduce potential delays in the treatment and increase survival rates.

Existing KPI-based assessments are typically performed for specific clinical departments, e.g. for the radiology department or for the surgery department, and/or for specific functional departments, e.g. financial, clinical, or administrative. However, these performances are interrelated, and overall patient satisfaction can be adversely impacted by poor performance in any area encountered by the patient.

It is often the case that EMR systems are composed of silos containing heterogeneous clinical, administrative, operational and financial information spread across several modules or subsystems. The use of sparse and fragmented pieces of information in KPI analytics jeopardizes their utilization as an actionable information source. When analyzed without considering the context of the whole healthcare enterprise (e.g., using only financial or only clinical data), KPIs may not provide comprehensive assessment of institution metrics, being unable to reveal important insights for the institution. For example, if an analyst in the hospital looks only at the billing data and realizes that the monthly income target will not be met, he/she might not be able to suggest corrective actions. These actions might depend on the understanding of several financial but also clinical and operational factors, such as the number of surgeries scheduled, the complexity of the procedures, the comorbidities found in the patient population, etc. Therefore, to analyze effectively healthcare data, one should be able to have a holistic view of the clinical, operational and financial events happening with the patient population of the healthcare institution.

The following discloses a new and improved systems and methods that address the above referenced issues, and others.

### SUMMARY

In one disclosed aspect, a health care performance assessment apparatus includes at least one processor programmed to: collect information associated with medically-related events from a plurality of databases; group the collected information associated with medically-related events into episode of care (EOC) data structures wherein each EOC data structure contains the collected information for a single EOC treating a medical condition or providing a medical procedure to a single patient; store the EOC data structures in an EOC repository; group EOC data structures having a chosen commonality into at least one cohort; and calculate at least one key performance indicators (KPIs) for the cohorts. A display is configured to display the KPIs for at least one selected cohort.

In another disclosed aspect, a non-transitory storage medium stores instructions readable and executable by one or more microprocessors to perform a method. The method includes retrieving information associated with a plurality of medically-related events; storing information related to the medically-related events; grouping the medically-related events into episodes of care each corresponding to a single patient; group the episodes of care into a plurality of cohorts based on similarly related episodes of care; calculate at least one KPI for the cohorts; and display the KPIs.

One advantage resides in generating KPIs to determine the clinical, financial, and operational resources for a healthcare treatment plan.

Another advantage resides in navigating displayed KPIs to determine a healthcare treatment plan.

Another advantage resides in analyzing multiple options of KPIs to determine a healthcare treatment plan.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a healthcare treatment planning apparatus for determining a healthcare treatment plan as disclosed herein.

FIG. 2 schematically illustrates a floor plan of a health institution using a display of the apparatus of FIG. 1.

FIG. 3 schematically illustrates one or more KPIs of the floor plan of FIG. 2.

FIG. 4 schematically illustrates a floor plan with at least one episode of care pathway of FIG. 3.

FIG. 5 schematically illustrates one or more cohorts associated with the episode of care pathways of a floor plan of FIG. 4.

FIG. 6 schematically illustrates one or more KPIs associated with the cohorts of FIG. 5.

FIG. 7 schematically illustrates one or more KPIs associated with the multiple cohorts of FIG. 6.

FIG. 8 schematically illustrates the KPIs associated with a selected one of the cohorts of FIG. 7.

FIG. 9 illustrates an exemplary flow chart for a method of use of the apparatus of FIG. 1.

### DETAILED DESCRIPTION

As previously mentioned, record keeping systems for medical institutions are commonly fragmented into various clinical, administrative, and financial record-keeping systems. Even if these diverse systems are integrated in the sense of data being transferrable from one system to another, this does not resolve the difficulty in leveraging the data for KPI analytics. Data may be stored in different formats, making their combination difficult. More fundamentally, there is no apparent linkage between clinical, administrative, and financial records. Thus, it is difficult to visualize how, for example, performance of the radiology department impacts in-patient hospitalization administration; or, how obtaining reimbursement pre-approval from an insurance company for a surgical procedure impacts scheduling of surgical procedures.

The present disclosure relates to generating Key Performance Indicator (KPI) metrics for a medical institution on-demand, using the “Episode of Care” (“EOC”) to account for interrelatedness of different departments of a medical institution, and in particular for determining relationships between clinical, administrative, and financial aspects.

To improve population health management and the quality of healthcare, the present disclosure describes an apparatus to present relevant clinical, operational and financial information for hospital management and decision making. This context-sensitive apparatus uses a data-driven strategy to explore healthcare KPIs by capturing all the events related to the healthcare process in the context of an EOC and providing on-demand information contextualized by the EOC. Taking the episode of care as a crucial backbone of information, the apparatus shows gradual exposure to performance information based on the medical professional needs and connected to the hospital and patient contexts. Furthermore, by providing near-real time data analytics, the apparatus empowers hospital decision makers with the right information at the right time.

The present disclosure provides a way to visualize and assess healthcare performance using an EOC framework. It is recognized herein that, although each individual patient will experience a unique EOC, it is possible to generate an “average” or “typical” episode of care by grouping medically-related events into EOC data structures and then grouping EOC data structures by some chosen commonality to define a “cohort” of EOC data structures. In this way, different sectors of the hospital can visualize the information using the EOC cohort, and have this information available for decision-making in real-time. Based on the chosen commonality, a cohort can group together EOC data structures in different ways. For example, a hospital medical manager can access the occupation rate information and act accordingly to increase or decrease occupation, without having to wait for long periods to obtain consolidated information, and then realize that the hospital has been under (or over) occupied in the previous period. Moreover, by being rooted on the EOC data structure which combines clinical, administrative, and financial data for the EOC of a single patient, the hospital manager can seamlessly correlate this KPI information to financial outcomes (e.g., under occupation might be the reason for reduced income) and to the patient satisfaction (e.g., over occupation of the hospital might have increased the waiting queues, negatively influencing patients' perceptions) to better understand the reasons for and impacts of the given KPI. Furthermore, by linking all the information to and categorizing the episodes of care into cohorts based on a chosen commonality, the apparatus allows analysts to easily come up with data-driven hypotheses for possible healthcare performance issues. This is possible by comparing cohorts that performed better to those that performed worse and identifying differences in the treatment between them. Hence, the apparatus targets the needs for holistic analyses, providing an atomic information source in the form of the EOC data structure which provides concise information for different sectors of the institution and allows comparisons between good and bad performing cohorts.

The apparatus leverages the EOC data structure to provide a connective framework for providing KPI metrics that account for these interrelations. The apparatus includes a data extractor which compiles relevant information on medically related events from various hospital databases, and possibly also from some private sources, in a standardized format such as using the JavaScript Object Notation (JSON) syntax. These data are grouped by an EOC builder to reconstruct EOC instances. Each EOC instance is represented by a data entity, referred to herein as an “EOC data structure” that is stored in an EOC repository and represents the EOC. Data elements of the EOC data structure are suitably labeled by department or the like, and information on medically related events in the EOC data structure are linked together by a time sequence for the events (except for non-event data entities having no time stamp, such as basic patient demographic data).

Each EOC corresponds to the services provided to a single patient to perform a medical procedure or to treat a medical condition. Data entities may be linked to a particular EOC in various ways, such as directly by referencing an EOC number stored in the patient EMR, or based on patient ID and a time interval bounded by hospital admittance/discharge or by a diagnosis time and a known treatment time frame or so forth. Some EOC may be unbounded (e.g. treatment for a chronic condition may have no end time).

The constructed EOC data structures serve as the basic data unit for KPI analysis by the apparatus. To perform a KPI analysis, an EOC cohort builder employs a classifier or other selection mechanism to select a cohort of EOC data structures that meet a chosen selection criterion (i.e. that have a chosen commonality). A KPI query engine applies a query to group EOC data structures having the chosen commonality together as a cohort and to compute one or more KPI metrics for the cohort.

A KPI viewer displays the KPI metrics for the cohort in a readily comprehensible fashion. In many cases, the cohort is selected in terms such that the patients of the cohort all follow a single pathway through the hospital, or at most a few alternative pathways. In some embodiments, the viewer displays this path in a graphical format (e.g. with nodes representing medically related events such as admission, radiology, surgery, and so forth) with which most or all patients of the cohort engage, and the KPI metrics are annotated to the various nodes. Alternatively, as most medically related events occur at various departments or agencies of the hospital having physical locations, the medically related events can be represented at their physical locations in a floor plan model of the hospital. This enables a hospital administrator to see precisely where (if anywhere) along the (average) episode of care the experience of the cohort is poor.

In general, the disclosed apparatus provides a way to review/synthesize/analyze historical hospital performance as assessed by the KPI metrics. In most tasks, the KPI metrics are computed for multiple patients forming a cohort. In some contemplated instances, however, the apparatus is applied to a single patient (i.e. a cohort of one) in order to assess a single patient's experience. This could be used, for example, by a nurse or other hospital employee to assist a patient who is lodging a complaint.

As used herein, the term “episode of care” (EOC) and variants thereof refers generally to all services (medical, financial, operational) provided to a single patient to treat a medical condition or to perform a medical procedure. Some non-limiting illustrative examples follow. An illustrative EOC encompasses all services provided to a patient from admission into the hospital with chest pains, through diagnosis of a heart attack, treatment provided for the heart attack and ancillary or contributory conditions such as high blood pressure, through discharge from the hospital and follow-up care for a specified time interval, e.g. 30 days following discharge. The services include medical services (e.g. physical examination in the emergency room and any follow-up physical examinations, any administered drugs, etc), operational services (e.g. providing an in-patient hospital room and any associated non-therapeutic services such as room cleaning/maintenance, meal services, television services, disposal of tissue excised during surgery, etc), and financial services (e.g. interactions with insurance companies). Another illustrative EOC encompasses all services provided in performing a hip replacement including medical services (initial physical examination, measurement and sizing and/or fabrication of the prosthetic hip, the hip replacement surgery, follow-up examination, removal of stitches, follow-up out-patient doctor's visit, physical rehabilitation therapy sessions), operational services (in-patient hospital room stay pre- and post-surgery and ancillary services), and financial services (obtaining pre-surgery insurance company approval, submitting insurance claims). The precise scope of an EOC may vary from embodiment to embodiment, and a given embodiment of the disclosed healthcare performance assessment tool may define an EOC more narrowly or more broadly. For example, a hospital employing the tool to assess its performance in providing in-patient care may define an EOC to have its initial boundary defined by patient admission and its ending boundary defined by patient discharge; whereas, a medical institution that provides a wide range of in-patient and out-patient services may more broadly define an EOC as extending from initial diagnosis of a medical condition or initial determination that the patient should undergo a medical procedure through hospital admission and discharge through follow-up care including subsequent out-patient doctor visits and rehabilitative physical therapy. As another example, an urgent care facility may define the EOC as beginning with a visit to the urgent care facility and ending with either being sent home or to a hospital for admission.

In the case of a chronic medical condition, the end of an EOC may be defined in terms of stabilization of the condition and return of the patient to a “normal” life within the constraints imposed by the chronic medical condition. Alternatively, in the case of a chronic medical condition the EOC may be defined as open-ended, i.e. having no defined end but rather continuing indefinitely as the patient continually receives therapy. The definition of an EOC may also vary from embodiment to embodiment in terms of how interrelated conditions are treated. For example, a hospital employing the tool for in-patient care assessment may define an EOC as the treatment of a heart attack; whereas, a medical institution focusing on a broader scope of care may define the EOC as encompassing treatment of contributory conditions such as high blood pressure, obesity, and smoking. A given embodiment may also employ a definition of the EOC promulgated by an outside agency such as an insurance company or a government (e.g., in the United States a definition of “EOC” for certain purposes is given in 32 U.S.C. § 1395cc-4(a)(2)(D) as meaning “with respect to an applicable condition and an applicable beneficiary, the period that includes—(I) the 3 days prior to the admission of the applicable beneficiary to a hospital for the applicable condition; (II) the length of stay of the applicable beneficiary in such hospital; and (III) the 30 days following the discharge of the applicable beneficiary from such hospital.”

The foregoing defines “EOC” in terms of medically-related events, where “medically-related” encompasses direct medical events (e.g. physical examination, surgery), ancillary treatment events (e.g. rehabilitative physical therapy), operational events (e.g. admission to a hospital, delivery of in-patient services and meal services), and financial events (e.g. obtaining pre-surgery insurance company approval, submitting insurance claims). In a data storage and data processing sense, the term “EOC data structure” denotes a data structure containing all information associated with the medically-related events collectively forming the EOC. In this sense, an EOC data structure may include, for example: patient demographic information such as age and gender; information on clinical events, including procedures, diagnoses, lab exams and medications, such as radiology reports, medical prescriptions, the patient's electronic medical record, or so forth; administrative and operational records such as dates of visits to specific locations (e.g. the radiology department), dates of hospital admission and discharge, in-patient hospital room information (location, identification of the assigned nurses and on-call physicians, etc); financial data such as insurance information, insurance claim records, billing information, and so forth. The EOC information may precede the earliest date of any medically-related event and/or may extend beyond the latest date of any medically-related event—for example, EOC information may include an insurance claim filed after discharge from the hospital even if that discharge date is taken as the end of the EOC, or similarly EOC information may include information about patient symptoms reported by the patient before hospital admission even if that admission is defined as the first medically-related event of the EOC.

The EOC data structure contains all these information. It is to be understood that the EOC data structure may “contain” these information directly, e.g. by the EOC data structure including copies of EOC-related information extracted from various databases, and/or indirectly, e.g. by the EOC data structure storing pointers or links to the information which is actually stored in some other database (e.g. the patient's electronic medical record, the billing department computer, or so forth). By way of illustration, a single patient may have a number of different EOC, e.g. one EOC for a heart attack, another EOC for a broken arm, and so forth. The patient's demographic information may be stored in a single location (e.g. the patient's electronic medical record) and each EOC data structure “contains” the demographic information by linking to that data in the patient's electronic medical record.

As used herein, the term “KPI” and variants thereof refers generally to at least one of clinical effectiveness, patient safety, efficiency, staff orientation, and governance for an episode of care.

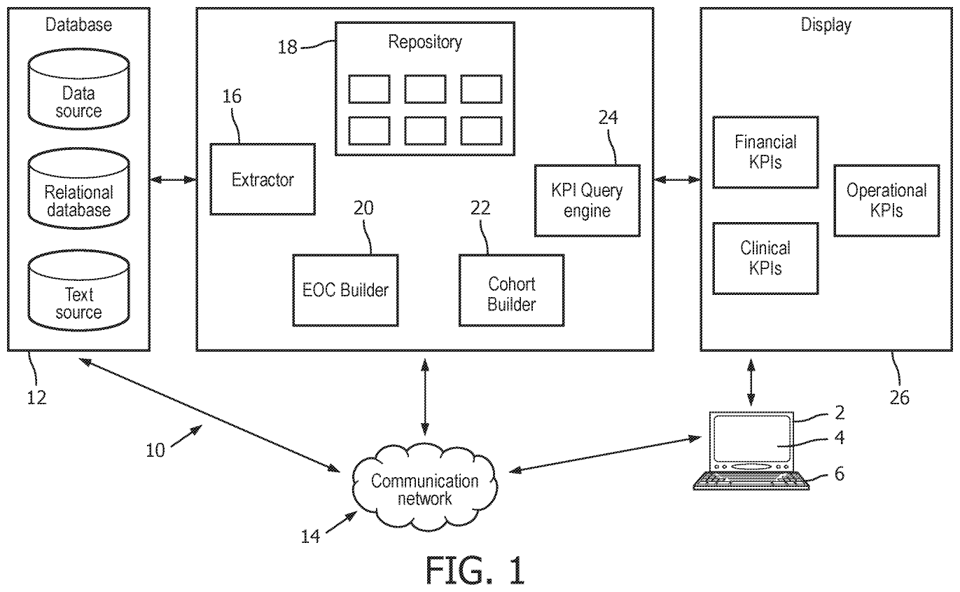

With reference to FIG. 1, in an exemplary embodiment a user (e.g. a hospital administrator) employs a user interface device 2, such as a computer with a display component 4 and at least one user input device 6 (e.g. an illustrated keyboard, and/or a mouse, trackball or other pointing device, and/or a touch-screen portion of the display component 4, or so forth) to interact with a healthcare performance assessment apparatus 10. The apparatus 10 is configured to extract and integrate relevant data needed to create aggregated, non-fragmented, and context-aware KPI views. The apparatus 10 integrates data from various healthcare data sources to provide a comprehensive understanding of patient population flows within the hospital and the metrics associated with them. The apparatus 10 is configured to captures relevant data related to patients and their medically related events, including clinical, financial and administrative events, and to group them into EOC data structures that serve as atomic pieces of information for the KPI analytics. Then, KPIs are computed using these episodes of care as source of information, usually by grouping EOC data structures that share a commonality chosen by the hospital administrator via the user interface device 2 into a cohort and computing KPIs for that cohort. By connecting the KPI metrics to the episodes of care (as represented by EOC data structures), at the population level (as represented by the cohort), the system allows the information to be better contextualized and thus understood. As shown in FIG. 1, the apparatus 10 can be implemented on a private cloud platform, such as a Hadoop ecosystem, to improve the storage and computing power thereof. Advantageously, the healthcare performance assessment device 10 includes one or more components that: (1) determine the clinical, financial, and operational resources for a healthcare treatment plan; (2) navigate displayed KPIs to determine a healthcare treatment plan; and (3) analyze multiple options of KPIs to determine a healthcare treatment plan.

As shown in FIG. 1, the apparatus 10 is in communication with a plurality of databases 12 containing information associated with medically related events. The databases 12 can include generic data sources, relational databases, text sources, or so forth containing information related to the episodes of care for patients of the hospital or other medical institution which is the subject of the KPI analytics apparatus 10. For example, the databases 12 can include information related to (i) patient demographics, including age and gender (usually contained in clinical, administrative, and financial databases); (ii) clinical events, including procedures, diagnoses, lab exams and medications (usually contained in the electronic patient record and/or other clinical databases); (iii) administrative and operational information, including the locations passed by in the institution, the respective time, the length of stay, and the treating physicians (these may be contained in human resources databases, departmental databases, a hospital-wide administrative database, or so forth); and (iv) financial data, including costs, health plans and billing (usually contained in a billing system, an admissions database, or the like). The apparatus 10 can be physically connected to the databases 12 (i.e., via a wired Ethernet, a USB cable or a cord and a corresponding port, or so forth), or electronically via a wired and/or wireless communications channel or network 14 (e.g., a wired and/or wireless Ethernet network, WiFi network, a local area network, a wide area network, the Internet, an intranet, a customer-supplied IEEE 802.11 wireless network, and the like).

The apparatus 10 includes an extractor 16 to extract and convert relevant information related of the episodes of care, a repository (or data storage) 18 to store episode of care information extracted from the hospital information systems; an episode of care builder 20 to reconstruct the episodes of care that aligns the information coming from several sources in order to generate EOC data structures representing the episodes of care; a cohort builder 22 to categorize the EOC data structures into different cohorts in accordance with chosen commonalities (e.g. defined categories); a KPI query engine 24 to provide a user-chosen commonality for which to build a cohort and compute and extract KPIs statistics for the cohort and generate a display 26 shown on the display component 2 of the user interface device 1 to map features of the episodes of care and KPIs to specific cost centers/departments within the healthcare provider. The display 26 optionally includes an interactive graphic user interface to display KPIs in the episode of care context. It will be appreciated that the apparatus 10 can add any desired components, or remove any of the shown components.

In some embodiments, the extractor 16 is programmed to retrieve information associated with a plurality of medically-related events. For example, the extractor 16 is in communication with the databases 12 via the network 14. The data coming from multiple data sources are highly heterogeneous, with different data types, data models, formats and semantics. The extractor 16 is configured to interface the different data sources with one or more homogenous programs (e.g., an application program interface (API) and one or more connection protocols). The extractor 16 is further configured to extract events from the databases 12 associated to at least one selected episode of care (e.g., treatment information for a heart attack). From the extracted data, the extractor 16 converts the different data into a single format, such as a single document model. For example, the extractor 16 converts the extracted data into a JavaScript Object Notation (JSON) format (or any other suitable syntax). The extractor 16 then transmits the single document model format of the data to the repository 18. The data streams are routinely loaded into the repository 18 using time stamps of the source data sets. Advantageously, the extractor 16 provides technical and syntactic interoperability for the apparatus 10.

The repository 18 is programmed to store all information related to a patient's episode of care found within the healthcare institution (i.e., in the databases 12). Alternatively, the repository 18 may store pointers or links to some such data which may remain physically stored at one or more of the databases 12. The repository 18 aggregates data from several healthcare data sources to create a unified register with the patient population flow, encoded in the episodes of care. The repository 18 is further programmed to receive the single document format of the data (i.e., in JSON format) that is extracted by the extractor 16 from the databases 12. In some embodiments, the repository 18 includes a NoSQL database, providing high model flexibility and retrieval performance. The NoSQL capabilities of the illustrative repository 18 allow the same to cluster data for retrieval, as described in more detail below.

In some embodiments, the episode of care builder 20 is programmed to link the different events belonging to a patient's episode of care and construct a data structure that stores (or associates) all this information together as an EOC data structure in the repository 18. For example, the episode of care builder 20 is programmed to retrieve at least one of the single document models for each of event of an episode of care from the repository 18. The episode of care builder 20 then links the different events together to form a data structure related to each event for an episode of care. In one example, the EOC data structure can be an array that includes all medically-related events for a single episode of care (e.g., the clinical procedures, locations, administrative aspects such as hospitalization and meal services, and financial costs or aspects such as billing and medical insurance reimbursement for a heart attack). The episode of care builder 20 then constructs an array for each episode of care. The episode of care builder 20 uses time and location features to organize the episode of care events in a meaningful way in the EOC data structure. Information associated with a particular EOC can be identified and reconstructed in various ways. This event identification and reconstruction is trivial in cases where data are associated to a common record identifier (i.e., primary key) corresponding to an EOC. However, due to the silo nature of healthcare data storage, it is often the case where a single key does not exist. For these cases, record linkage methods to link data medically related data to a single EOC are applied to connect and capture all care events. For example, such linkages may be made based on patient name or identifier (since each EOC is for a single patient), ICD-9 code (where, for example, a sub-class of ICD-9 codes are known to be directly or indirectly associated with treatment of a medical condition such as a heart attack), time frame (for example, if the delineation of medical events associated with an EOC is defined as being bounded by hospital admission and discharge), doctor's name (for example, any charges associated with a patient's pulmonologist within an EOC time frame may be reasonably assigned to an EOC data structure for an EOC treating a respiratory ailment), or so forth. In addition, medically related event data of the EOC data structure includes time stamps and can be arranged in the EOC data structure in a temporal sequence (except for data entities having no time stamp, such as basic patient demographic data). Once constructed, the episode of care builder 20 transmits the EOC data structures to the repository 18 for storage therein. (As previously noted, the EOC data structure may store the collected information associated with medically-related events of the EOC, and/or may store links or pointers to such information stored elsewhere such as in the databases 12—such information is deemed herein to be “contained” in the EOC data structure whether it is physically stored in the EOC data structure or stored via a suitable pointer or link.)

The cohort builder 22 is programmed to automatically create episode of care cohorts so that KPIs can be associated to certain patient groups, and root-cause analyses can be performed. For example, the cohort builder 22 is programmed to retrieve at least one EOC data structure, such as an EOC array, containing information related to an episode of care from the repository 18. From the retrieved EOC data structures, the cohort builder 22 generates at least one cohort by grouping EOC data structures that have a chosen commonality, such as a common medically-related event (e.g., the clinical procedures, locations, and financial costs for patients that have had a heart attack). In some instances, the cohort builder 22 includes one or more machine-learning algorithms (e.g., k-Nearest Neighbors, Support Vector Machines, Neural Networks, and the like). In other instances, the cohort builder 22 retrieves the episode of care data structures from the repository 18 for training and testing. Once the cohorts are generated, the cohort builder 22 transmits to the cohorts to: (1) the KPI query engine 24; or (2) the repository 18 for storage therein (the cohorts are depicted in FIG. 1 as boxes with in the repository 18).

In some embodiments, the KPI query engine 24 is programmed to calculate a plurality of KPIs and assess the same. For example, the KPI query engine 24 is programmed to either: (1) retrieve the generated cohorts from the repository 18; or (2) receive the generated cohorts directly from the cohort builder 22. The KPI query engine 24 then applies a query to the cohorts, and computes a plurality of KPI values from the cohorts. To do so, the KPI query engine 24 implements statistical functions (e.g., descriptive and/or predictive functions) to compute the different KPI values (e.g., the financial, clinical, and operational KPIs for instances of a heart attack). In computing a KPI, various aggregations over the EOC data structures of the cohort may be used as appropriate for the desired assessment. For example, if the goal is to assess the average length of hospitalization performance indicator for hip replacement patients, the KPI may be the average length of hospitalization (that is, the value of the performance indicator for a given EOC data structure) for all EOC data structures of a cohort defined by the chosen commonality of the patient having undergone hip replacement surgery. On the other hand, if the goal is to assess the total income performance indicator for treating heart attacks, the KPI may be the sum total of collected billings and granted insurance claims (the value of the performance indicator for a given EOC data structure in this case) for all EOC data structures of a cohort defined by the chosen commonality of the patient being admitted for treatment of acute myocardial infarction (AMI). Once the KPI values are generated, the KPI query engine 24 applies an interface mechanism to the KPIs. For example, the KPI query engine 24 provides a Representational State Transfer (REST) API or other web service to allow a medical professional to retrieve the KPIs, as described in more detail below. The KPIs can be retrieved based on one or more selected parameters, such as period (e.g., from and to date), type of analyses (e.g., mortality, length of stay, etc.), and stratification groups (e.g., gender and age). Once the KPIs are generated, the KPI query engine 24 transmits the same to the display 26.

It will be appreciated that the extractor 16, the episode of care builder 20, the cohort builder 22, and the KPI query engine 24 operate in real time. Advantageously, by providing real time data analytics, the apparatus 10 can empower hospital decision makers with the right information at the right time. In one approach, the extractor 16 updates the collecting of information, and the EOC builder 20 continually updates the grouping of newly collected data into EOC data structures (either adding the newly collected data to existing EOC data structures or creating new EOC data structures for new episodes of care, as appropriate), and the new or update EOC data structures are stored in the repository 18. By “continually” it is meant that such updates are performed on a regular basis, e.g. once per 24 hour period preferably at a specified time in the overnight hours. Additionally or alternatively, such updates can be driven by the source, e.g. whenever data in the databases 12 is updated this triggers operation of the extractor 16 and EOC builder 20 to add the data to the appropriate EOC data structure. In this way the repository 18 is kept up-to-date (possibly with a small latency, e.g. up to 24 hours in the case of overnight updating) so that the cohort builder 22 can group EOC data structures into a cohort and the query engine 24 can calculate KPIs for the cohort on-demand.

The display 26 is configured to display the KPIs for a medical professional to see and navigate. For example, the interface mechanism included in the KPIs by the KPI query engine 24 allows a medical professional, once the KPIs are displayed as an interface to visualize and navigate the KPIs to determine an optimal patient treatment plan (e.g., or treating patients having heart attacks). The medical professional can filter the KPIs based on one or more of time, location, demographics, clinical resources, operational resources, and financial resources. Such filtering may for example be implemented by adjusting the chosen commonality defining the cohort and updating the cohort appropriately and then re-computing the KPIs for the updated cohort. In one example, the display 26 can display at least one area of a medical care center. In another example, the display 26 can display at least one KPI for the at least one area of the medical care center. The interface can be created using a medical professional-centered design methodology and implemented using a protocol such as HTML5.

The various data processing components 16, 20, 22, and 24 are suitably implemented as a computer, computing system, cloud computing resource, microprocessor or the like that is programmed by software to perform the disclosed operations. To access the databases 12, the healthcare performance assessment tool 10 is preferably implemented in a networked environment with connection to the hospital data network, departmental computer networks (e.g. PACS system used by radiology), and optionally also with connection to outside data resources via the Internet. The networked environment also enables a hospital administrator or other user to access the healthcare performance assessment tool 10 via a computer or workstation 2 which may in general be located in the administrator's office or may be accessed remotely, e.g. using a Virtual Private Network (VPN) or the like. While a single user interfacing computer or workstation 2 is illustrated as an example, the healthcare performance assessment tool is preferably a multi-user tool that is accessible by various hospital administrators, medical professionals with administrative responsibilities, or other users. In some embodiments, users may be assigned authorization or access levels and the data presented in the display 26 may be filtered based on the user's access level so that the user can access only authorized data.

The various data processing components 16, 20, 22, and 24 of the apparatus 10 may also be implemented as a non-transitory storage medium storing instructions readable and executable by a microprocessor (e.g. as described above) to implement the disclosed operations. The non-transitory storage medium may, for example, comprise a read-only memory (ROM), programmable read-only memory (PROM), flash memory, or other repository of firmware for the apparatus 10. Additionally or alternatively, the non-transitory storage medium may comprise a computer hard drive (suitable for computer-implemented embodiments), an optical disk (e.g. for installation on such a computer), a network server data storage (e.g. RAID array) from which the apparatus 10 or a computer can download the system software or firmware via the Internet or another electronic data network, or so forth.

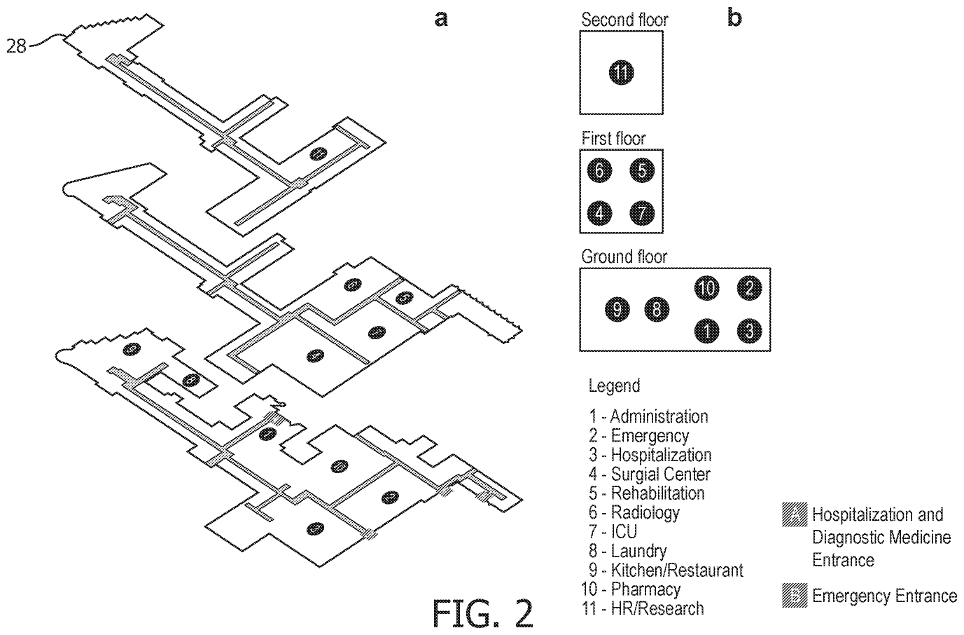

FIGS. 2-8 illustrate an example of navigating the KPIs shown in the display 26 to allow a medical professional to select the best health care plan for one or more patients. FIG. 2 shows a graphical visualization of a floor plan 28 of one or more healthcare institute locations (e.g., departments, sectors, specialties, and the like). FIG. 2 shows two panels of the floor plan, a detailed view (panel a), describing exactly the hospital physical locations, or a simplified view (panel b), so that visualization is improved. In one example, FIG. 2 can show a representation of a “welcome screen” for the medical professional, since the KPIs have not been displayed on the display 26 at this point. The floor plan 28 can include one or more rooms shown in the room legend 30 (e.g., an administration room 1, an emergency room 2, a hospitalization room 3, a surgical center 4, a rehabilitation room 5, a radiology room 6, an intensive care unit (ICU) room 7, a laundry room 8, a kitchen/restaurant room 9, a pharmacy 10, and a human resources/research room 12). Although the foregoing is discussed in terms of a treatment plan for a heart attack, it will be appreciated that the foregoing can also be used for any other suitable treatment plan.

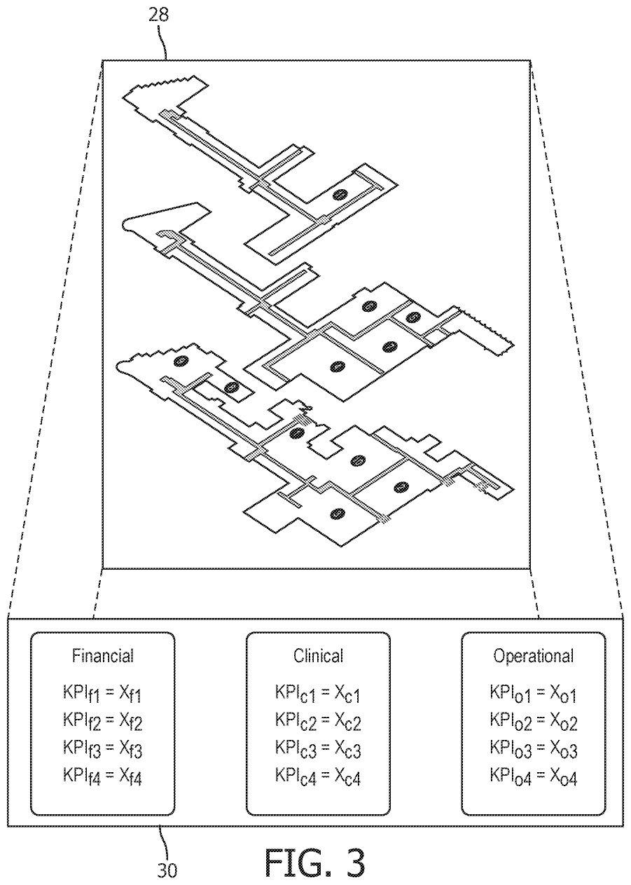

FIG. 3 shows a KPI list 30 for the floor plan 28. For example, the KPI list 30 can include one or more KPIs related to financial KPIs, clinical KPIs, and operational KPIs for a treatment plan (e.g., for treating patients having a heart attack). This view allows the medical professional to see a “snapshot” of KPIs prior to any patient treatment. The medical professional can also visualize KPI trends and correlation of different KPIs from the KPI list 30. Furthermore, the display 26 allows the medical professional to zoom into the different locations and get a similarly set of KPI views contextualized in that location (e.g., KPIs for the emergency room 2, the rehabilitation room 5, and the like). For example, the medical professional could zoom into the ICU unit and get the clinical, operational, and financial KPIs associated to this location and their trends for treating patients having a heart attack in that location.

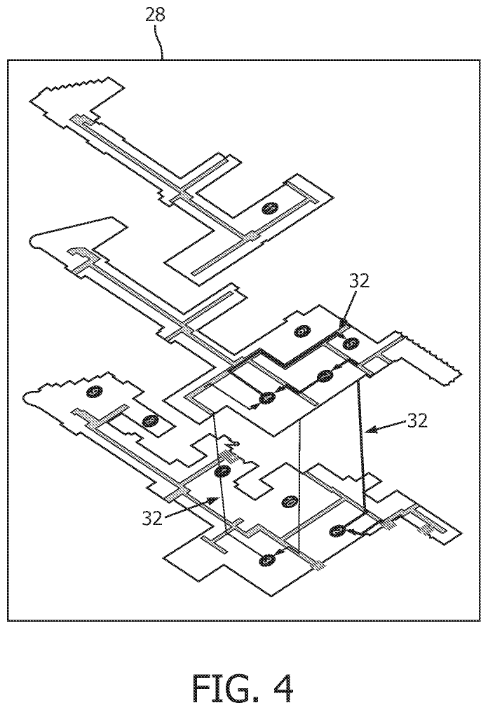

FIG. 4 shows a plurality of episode of care pathways 32 indicative of the KPI list (not shown in FIG. 4) for the floor plan 28. The episode of care pathways 32 shows the flow of patients within the institution for a given period for a medical event (e.g., a heart attack). As shown in FIG. 4, wider paths indicate larger flows while thinner paths indicate smaller flows between locations. These flows are determined from the cohort of interest retrieved from repository 18, taking into account the selection period or other chosen commonality defining the cohort. The pathway may be determined for a cohort based on the timeline of events for each cohort, using suitable averaging or other aggregation. For example, in FIG. 4 a primary example pathway 32 (i.e., the thicker pathway) shown in FIG. 4 for treating a patient having a heart attack includes transporting a patient from the emergency room 2 to the ICU 7 to the surgical center 4, and to the rehabilitation room 5. This pathway is illustrated as a wide path illustrating that a large fraction of patients of the cohort indicate follow this sequence of events. A secondary example pathway 32 (i.e., the thinner pathway) shows that some heart attack patients can be transferred to the hospitalization room 3 before being transferred to the surgical center, in cases where surgery is not immediately performed. This thinner pathway indicates that a smaller fraction of patients of the cohort follow this pathway.

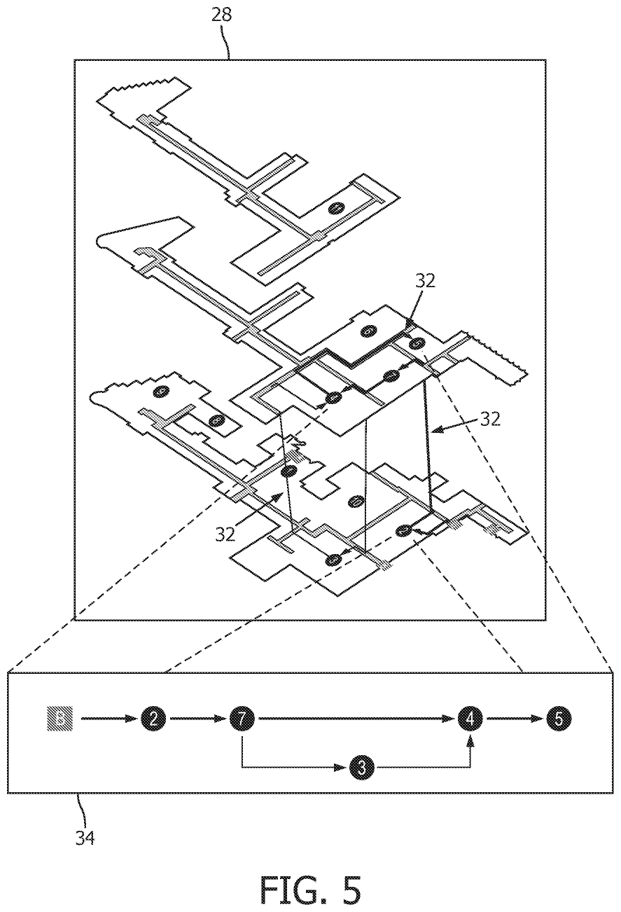

FIG. 5 shows an extracted view of the pathways 32. For example, the medical professional can select an episode of care-based filter view, allowing the medical professional to extract and focus on episode of care pathways of interest. The medical professional is able to filter the episode of care pathways 32 by time (period), location (e.g., by the department, specialty, etc.), demographics (e.g., gender, age), and clinical (e.g., procedure, diagnosis), operational (e.g., length of stay, occupation) and financial (e.g., cost, income) features (i.e. chosen commonalities) for a plurality of patients having, or already had, heart attacks (a further chosen commonality). FIG. 5 shows an example of filtering the episodes of care by location, where medical professionals have stayed in locations 2 and 5. For example, the pathway 32 described above in FIG. 4 (i.e., from the emergency room 2 to the ICU 7 to the surgical center 4, and to the rehabilitation room 5) shows the user the “stops” along the pathway 32. The secondary example pathway 32 (i.e., including a “stop” at the hospitalization room 3) is shown as an optional stop by “veering” off the primary pathway 32. The extracted list of episode of care is shown as a cohort list 34.

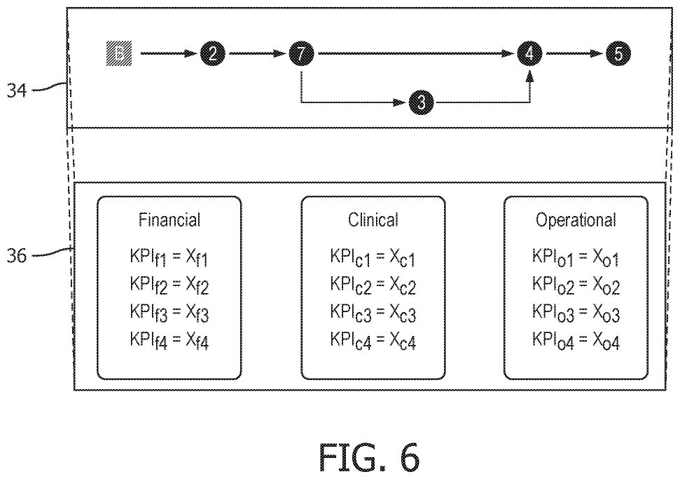

FIG. 6 shows a KPI list 36 associated with the cohort list 34 of FIG. 5. This view is similar to the view of FIG. 3. However, instead of being contextualized in the institution location, it is contextualized in the episodes of care cohorts 34 for heart attack treatment. This view allows the users to see KPI snapshots, as well as trends and correlations for heart attack treatment plans. The medical professional could get the clinical, operational and financial KPIs associated with each selected cohort list 34 and their trends.

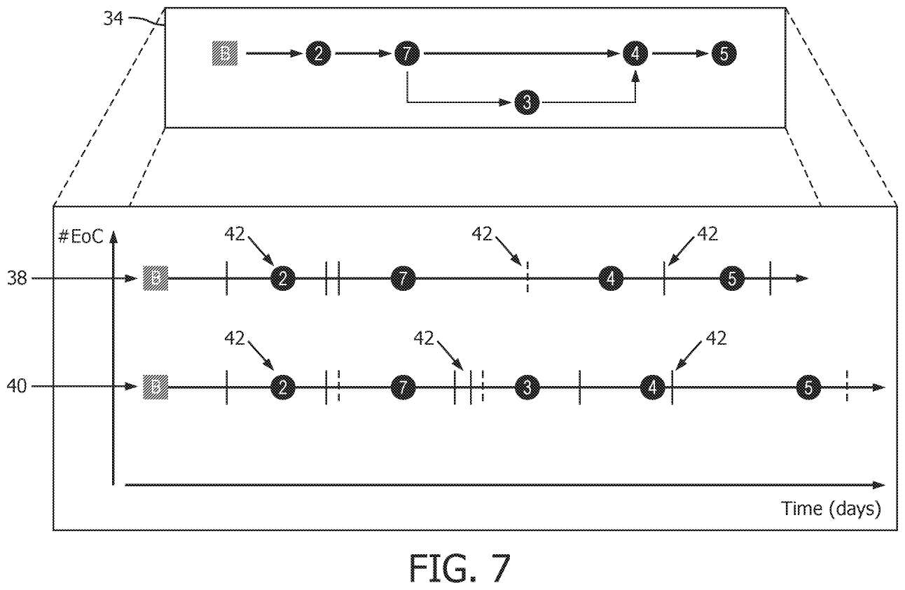

FIG. 7 shows an expanded view of the cohort list 34 of FIG. 6. The cohort list 34 is displayed as a primary cohort list 38, which includes the cohorts for the primary episode of care pathway 32 (i.e., rooms 2, 7, 4, and 5), and a secondary cohort list 40, which includes the cohorts for the secondary episode of care pathway (i.e., rooms 2, 7, 3, 4, 5). The medical professional can also visualize the KPIs 42 (shown schematically as solid or dashed lines) for each cohort list 38 and 40. For example, the solid lines 42 can depict financial KPIs, while the dashed lines 42 can symbolize clinical KPIs.

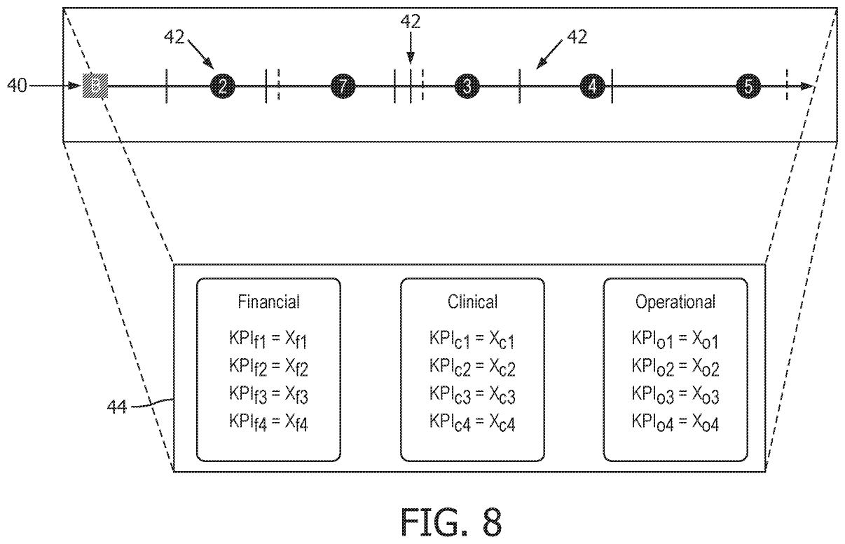

FIG. 8 shows a KPI list 44 for a selected one of the primary and secondary cohort lists 38 and 40. For example, the medical professional can select one of the cohort lists 38 and 40 (in this case, the secondary cohort list 40). Upon selection, the display 26 can display the KPI list 44 for the secondary cohort list 40. Similar to the KPI lists 30 and 36, the KPI list shows the clinical, operational and financial KPIs associated with the selected secondary cohort list 40 and their trends. Similarly, although not depicted, the medical professional can select the primary cohort list 38 to see the associated KPIs of the primary cohort list. From the KPIs, the medical professional can determine the optimal cohort, and thus the best healthcare treatment plan for the patient (e.g., for heart attack treatment). For example, the cohort list 38 and 40 with the highest KPI values is selected as the best healthcare treatment plan for the patient.

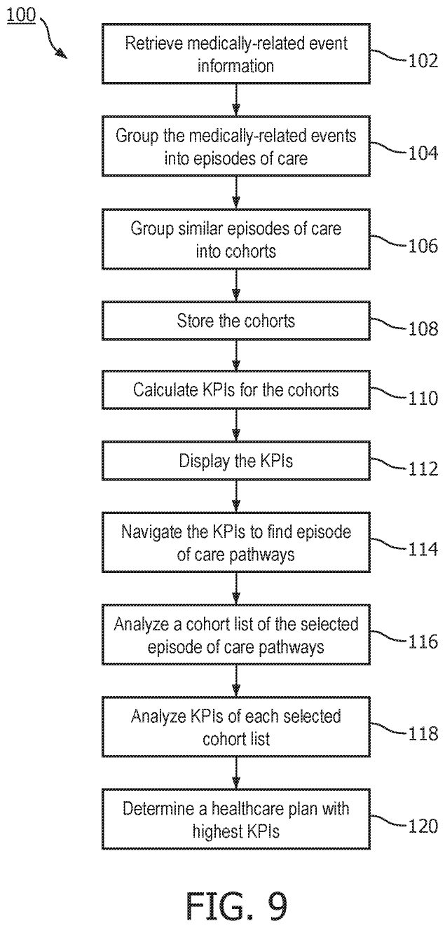

FIG. 9 shows an exemplary flow chart of a method 100 of using the healthcare performance assessment apparatus 10 of FIG. 1. The method 100 includes the steps of: retrieve information associated with a plurality of medically-related events (Step 102); group the medically-related events into episode of care data structures (EOC data structures) each corresponding to a single patient (Step 104); group the EOC data structures into a plurality of cohorts based on similarly related episodes of care (Step 106); store the cohorts in an EOC repository (Step 108); calculate at least one KPI for the cohorts (Step 110); display the KPIs (Step 112); navigate the KPIs to find one or more episode of care pathways (Step 114); analyze a cohort list associated with each selected episode of care pathways (Step 116); analyze one or more KPIs associated with each selected cohort list (Step 118); and determine a healthcare plan with the highest KPIs for the selected cohort list (Step 120).

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A health care performance assessment apparatus comprising:
at least one processor programmed to:
collect information associated with medically-related events from a plurality of databases;
group the collected information associated with medically-related events into episode of care (EOC) data structures wherein each EOC data structure contains the collected information for a single EOC treating a medical condition or providing a medical procedure to a single patient;
store the EOC data structures in an EOC repository (18);
group EOC data structures having a chosen commonality into at least one cohort;
calculate at least one key performance indicators (KPIs) for the cohorts; and
a display configured to display the KPIs for at least one of the cohorts;
wherein the at least one processor is programmed to construct the display of the KPIs for the cohort as a EOC pathway for the cohort with nodes corresponding to medically-related events and connections corresponding to transfers of patients of the cohort between medically related events and with at least one of nodes and connections being labeled with the calculated KPIs for the cohort.

2. (canceled)

3. The apparatus according to claim 1, wherein the EOC data structures include event timelines constructed using timestamps of the medically-related events whose information is collected in the EOC data structures, and the EOC pathway is constructed for the cohort by aggregating event timelines of the EOC data structures of the cohort.

4. The apparatus according to claim 1, wherein the at least one processor is programmed to calculate a key performance indicator (KPI) for the cohort by aggregating the values of the performance indicator for the EOC data structures of the cohort.

5. The apparatus according to claim 4, wherein the aggregating is one of averaging and summing the values of the performance indicator for the EOC data structures of the cohort.

6. The apparatus according to claim 1, wherein the chosen commonality comprises one or more chosen commonalities selected from a group consisting of time period, location, service by a chosen medical department, patient demographic data, medical procedure or medical condition, length of hospitalization, and financial data.

7. The apparatus according to claim 1, wherein the KPIs are indicative of at least one of clinical effectiveness, patient safety, efficiency, staff orientation, and governance for an episode of care.

8. The apparatus according to claim 1, wherein the at least one processor is further programmed to update the collecting of information, the grouping into EOC data structures, and the storing continually whereby the operations of grouping EOC data structures into a cohort and calculating KPIs for the cohort can be performed on-demand.

9. The apparatus according to claim 1, wherein the data contained in the EOC data structure includes at least:
(i) patient demographics, including age and gender;
(ii) clinical events, including procedures, diagnoses, lab exams and medications;
(iii) administrative and operational information, including the locations passed by in the institution, the respective time, the length of stay, and the treating physicians; and
(iv) financial data, including costs, health plans and billing.

10. The apparatus according to claim 1, wherein the at least one processor is further programmed to:
convert the extracted data from the plurality of databases into a single document model format for each event of an episode of care;
retrieve at least one of the single document models for each of event of an episode of care from the data storage;
link the different events together to form the EOC data structure related to each event for an episode of care with time stamped events of the EOC data structure linked in a temporal sequence by the timestamps; and
transmit the EOC data structure to the data storage.

11. The apparatus according to claim 1, wherein the at least one processor is further programmed to:
retrieve at least one data structure related to each event in an episode of care from the data storage;
generate at least one cohort from the data structures by grouping episodes of care that have a common medically-related event; and
at least one of
transmit the cohorts to another processor of the at least one processors; and
transmit the cohorts to the data storage.

12. The apparatus according to claim 1, wherein the at least one processor is further programmed to:
retrieve at least one cohort;
compute a plurality of KPI values from the cohorts;
apply an interface mechanism to the KPIs; and
transmit the KPIs to the display.

13. The apparatus according to claim 1, wherein the display is further configured to:
allow a medical professional to visualize and navigate the KPIs to determine an optimal patient treatment plan, the medical professional being able to filter the KPIs based on one or more of time, location, demographics, clinical resources, operational resources, and financial resources; and
display at least one area of a medical care center;

14. The apparatus according to claim 13, wherein the display is further configured to:
display at least one KPI for the at least one area of the medical care center.

15. The apparatus according to claim 1, wherein the at least one processor is further programmed to operate in real-time.

16. A non-transitory storage medium storing instructions readable and executable by one or more microprocessors to perform a method, comprising:
retrieve information associated with a plurality of medically-related events;
store information related to the medically-related events;
group the medically-related events into episodes of care each corresponding to a single patient;
group the episodes of care into a plurality of cohorts based on similarly related episodes of care;
calculate at least one KPI for the cohorts; and
display the KPIs
wherein the one or more microprocessors is programmed to construct the display of the KPIs for the cohort as an EOC pathway for the cohort with nodes corresponding to medically-related events and connections corresponding to transfers of patients of the cohort between medically related events and with at least one of nodes and connections being labeled with the calculated KPIs for the cohort.

17. (canceled)

18. (canceled)

19. (canceled)

20. (canceled)

21. (canceled)

22. (canceled)

23. (canceled)

24. (canceled)

25. (canceled)
